(19) United States
(12) Patent Application Publication
(10) Pub. No.: US 2011/0224637 A1
(43) Pub. Date: Sep. 15, 2011

(54) LOW pH, OPTIMAL ORP, AND ODOR-REDUCING FIBERS, A PROCESS FOR MAKING THE FIBERS, AND ARTICLES MADE THEREFROM

(75) Inventors: Keith EDGETT, Middletown, DE (US); Robert JORGENSEN, Middletown, DE (US); Eugene P. DOUGHERTY, Camdenwyoming, DE (US)

(73) Assignee: Playtex Products, LLC

(21) Appl. No.: 13/028,839

(22) Filed: Feb. 16, 2011

Related U.S. Application Data
(60) Provisional application No. 61/338,195, filed on Feb. 16, 2010.

(51) Int. Cl. A61F 13/20; B01J 20/22; B01J 20/26

(52) U.S. Cl. 604/358; 502/401; 502/402

(57) ABSTRACT

The present disclosure provides low pH fibers that are treated with additives, preferably after regenerating the cellulosic fiber, to control the pH and oxidation-reduction potential (ORP) in an aqueous environment where the fiber is placed. The low pH fibers can be formed into fibrous articles such as tampons or wipes. The low pH fibers with the additives provide health benefits to the user in that they hinder the ability of harmful bacteria to flourish.

### CROSS-REFERENCE TO RELATED APPLICATIONS

The present application claims the benefit of U.S. Provisional Application No. 61/338,195, filed on Feb. 16, 2010.

### BACKGROUND OF THE DISCLOSURE

1. Field of the Disclosure

The present disclosure relates to fibers that can lower pH and influence oxidation-reduction potential in the environment in which they are used. The fibers can be used in absorbent articles, such as wipes and tampons.

2. Discussion of the Related Art

Generally, the pH of the vagina is maintained between 4 and 4.5. Vaginal discharges are acidic, and that acidity has been linked to the presence of gram-positive organisms, such as the Doederlein bacteria. The “sour environment” in the vagina tends to promote growth of lactic acid bacteria that can multiply and survive best at a low pH (i.e. pH in the 4-4.5 range). The increase in lactic acid bacteria colonies compete for nutrients, thus preventing other, more harmful bacteria from growing, and thereby protecting the body from unpleasant infections and inflammations.

Most pathogenic bacteria and fungi have a fairly restricted pH range. Generally speaking, harmful bacteria and fungi tend to multiply, grow and survive best at a pH of about 7.5. The pH of blood is typically 7.2 to 7.7, much higher than that of normal vaginal secretions. Thus, not surprisingly, during a woman's period, the pH of her menstrual fluid rises typically to a range of about 6.9 to 7.2. After intercourse, the pH of a woman's vagina typically increases as well, since the pH of semen ranges typically from 7 to 8.

The optimal growth of certain pathogenic bacteria is also related to the electrochemical enzymatic reactions concerned with nitrogen metabolism. Thus, oxidation-reduction potential (ORP) is important to vaginal health as well. To prevent growth of pathogenic bacteria and to promote overall vaginal health, an antioxidant environment is preferred; that is, the environment should be more “reducing”, exhibiting a low ORP. ORP vaginal control would certainly make sense, since menstrual and vaginal fluid samples from women typically are comprised of elements that can take on different valency levels, e.g. copper, zinc, iron, and chlorine. Moreover, electron transport mechanisms in cells are often primarily electrochemical in nature.

ORP values should range between certain limits. Generally, ORP values are expressed in millivolts (mv). The CRC Handbook lists the electrochemical series for half-reactions that range from −3100 my up to 3030 my. Very strongly reducing values of ORP are highly negative numbers (<−1500), while strongly oxidizing values are large positive numbers (>1200). For the most part, ORP values in biological systems should be slightly negative values (i.e. having some reducing or anti-oxidant capability).

Furthermore, many women have problems with menstrual odors. Causes can be bacterial vaginosis, yeast (fungal) infections; Trichomonas (a different bacterial infection), sweating, urine, a retained tampon, and each women's unique individual scents. Many women have a condition known as “fish-odor syndrome”, also known as trimethylaminuria, an inherited metabolic disorder in which affected individuals excrete excessive amounts of the naturally occurring tertiary amine trimethylamine in their breath, sweat, urine and other bodily secretions. This amine smelly strongly of rotting fish and is a powerful neurolfactant readily detected by the human nose at very low concentrations, even at less than 1 part per million.

With skin, a healthy pH has been assessed to be between 4.5 and 6.0. Numerous skin care products, such as baby products, are formulated at a pH of about 5.5. Most commercial antibacterial wipes products are formulated with a solution exhibiting a pH of about 4.5. It is commonly believed that having personal care products matching the average pH of skin might be beneficial to skin's health, by maintaining the skin at its normal pH. The skin of babies, however, is often exposed to feces and urine residues, which push up the pH on the baby's skin above desired values. Similar to what is discussed above, this can lead to an environment where harmful bacteria can thrive.

Accordingly, there is a need for devices, such as absorbent articles like tampons and wipes, which can affect both pH, ORP, and vaginal odor to advantage.

### SUMMARY OF THE DISCLOSURE

The present disclosure provides fibers, and a method for making the same, that can provide lower pH, ORP control, and reduce odor when the fibers are in an aqueous environment. The fibers can be incorporated into fibrous articles for use, such as a catamenial tampon or other articles used by women in the vaginal area, or in a wipe.

The fibers are treated with several additives, as discussed below, to achieve the lower pH, ORP, and odor reduction. The additives comprise an acid, which can be present alone or in conjunction with the corresponding mineral salt of the acid, a finishing agent, and several optional additional components. The present disclosure has discovered that this particular combination has been effective at lowering pH, influencing ORP, and reducing odor in aqueous environments.

The fibers of the present disclosure, when placed in an aqueous solution, such as that found in the vaginal area of women, impart a pH of about 3.5 to about 4.7 to the solution when measured according to the method described below. In another embodiment, the resulting pH of the aqueous solution can be from about 3.7 to about 3.9. The fibers also impart favorable ORP characteristics to that solution, and help to reduce odor. As discussed above, this provides numerous benefits to a user, since it can keep harmful bacteria and harmful fungi from thriving.

Thus, in one embodiment, the present disclosure provides a low pH fiber, comprising about 0.05% to about 0.8%, based on the weight of the fiber, of a first additive selected from certain acids, and about 0.08% to about 0.7% of a second additive selected from certain finishing agents.

In a second embodiment, the present disclosure provides a fibrous article. The fibrous article comprises a plurality of low pH fibers, about 0.30% to about 0.65%, based on the weight of said plurality of low pH fibers, of a first additive selected from certain acids and salts thereof, and about 0.08% to about 0.7%, based on the weight of said plurality of low pH fibers, of a second additive selected from certain finishing agents.

In another embodiment, the present disclosure provides a process for making a low pH fiber. The process comprises the steps of converting natural cellulose to cellulose xanthogenate, dissolving the cellulose xanthogenate in alkali, to form a colloidal viscose, coagulating the colloidal viscose, drying the colloidal viscose, drawing a fiber from the dried colloidal viscose, and adding an acid selected from the group consisting of citric acid, lactic acid, isoascorbic acid, and any combinations thereof, to either the colloidal viscose, or the fiber, to form the low pH fiber.

### BRIEF DESCRIPTION OF THE DRAWINGS

FIG. 1 is a plot of the concentration of lactic acid versus pH in a fiber of the present disclosure; and

FIG. 2 is a cross-sectional view of a fiber of the present disclosure.

### DETAILED DESCRIPTION OF THE DISCLOSURE

As discussed above, several additives are added to the fiber, to achieve low pH and ORP control when the fibers are in an aqueous environment. These additives include acids, alone or in conjunction with mineral salts thereof, and finishing agents. In one embodiment, the acids and optionally the mineral salts thereof are added after the cellulose has been regenerated from viscose. Other optional ingredients may also be added to the fibers, including but not limited to non-acids and encapsulating agents. The order of addition of these optional additive chemicals may vary slightly. Additions are usually made before the fiber is cut to size, but could be done after this.

### A. Acids

One of the additives used to treat the fibers is an acid, alone or in combination with a mineral salt thereof. The present disclosure uses the term “mineral salt” to indicate a salt of the chosen acid that would produce the same anion as the acid, and thus provide a pH buffering effect. Examples of metals that are suitable candidates for the mineral salts of the present disclosure are, but are not limited to, sodium, potassium, calcium, and other alkali and alkaline earth metals. Thus, in one example, when the chosen acid is lactic acid, suitable mineral salts thereof would include sodium lactate and potassium lactate.

The acid can be citric acid, lactic acid, isoascorbic acid, glycolic acid, malic acid, tartaric acid, glycolide (a cyclic dimer or a glycolic acid which hydrolyzes to form two glycolic acid molecules), acetic acid, dehydroacetic acid, boric acid, oleic acid, palmitic acid, stearic acid, behenic acid, palm kernal acid, tallow acid, salicylic acid, ascorbic acid, sorbic acid, benzoic acid, succinic acid, acrylic acid (including its polymeric forms: polyacrylic acid as well as sodium polyacrylate buffer), or any combinations thereof. As previously discussed, acids and mineral salts of the corresponding acids could be added together (e.g. lactic acid and sodium lactate) to provide a “buffering” effect, which helps keep the pH stable over the environmental exposures that the fiber may encounter over time. Preferred acids include citric acid, lactic acid, isoascorbic acid or any combinations thereof. Of these, lactic acid together with a corresponding mineral salt, such as sodium lactate or potassium lactate, is most preferred, since the Lactobacillus acidophilus bacteria, present in normal, healthy vaginal flora, produces lactic acid as a metabolic by-product. Acids like lactic acid, citric acid, and isoascorbic acid are also preferred because, being somewhat hydrophobic, they will be less likely to be washed out by water treatments commonly performed during fiber and nonwoven processing.

A more complete list of potential acids that can be used appears in the CRC Handbook, in particular sections D129 and D130, which are herein incorporated by reference. (CRC Handbook of Chemistry and Physics, 54^{th} Edition, Cleveland, Ohio: CRC Press, 1973-1974.) Generally, the preference would be for organic, weakly acidic materials (i.e., where 2<pKa<6), since the preferred vaginal pH range is 3 to 7, 3.5 to 5.5 being more preferred. Very strong acids are generally less preferred, because unless these acids are very highly diluted, the pH can be dangerously low, adversely affecting rather than promoting vaginal health. Without being bound by theory, it is believed that the acids suitable for treatment of the fibers of the present disclosure act in the body as an antioxidant, attacking free radicals that might otherwise promote growth of certain pathogenic bacteria, fungi, protozoa, etc.

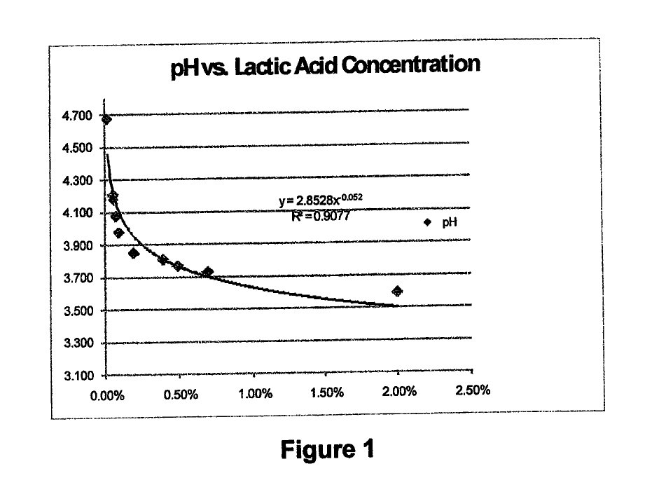

The acid, alone or in conjunction with the mineral salt thereof, is present in an amount of about 0.05% to about 0.8%, or between exactly 0.05% and exactly 0.8%. In one embodiment, the acid is present in an amount of about 0.30% to about 0.65%, or between exactly 0.30% and exactly 0.65%. In another embodiment, the acid is present in an amount of about 0.40% to about 0.60%, or between exactly 0.40% and exactly 0.60%. These weight percentages are based on the weight of the fiber, or in the case where there is a plurality of fibers in an absorbent article, on the weight of the total fiber content of the article. FIG. 1 shows a plot of how the amount of acid affects the pH of the fiber in the article, when the acid is lactic acid.

In addition to the weak acids and the mineral salts thereof discussed above, some salts could be added alone to adjust pH and ORP. For example, salts of weak bases, such as ammonium chloride, ammonium bromide, benzalkonium chloride, palmitamidopropryltriammonium chloride and similar salts of weak bases can undergo hydrolysis reactions with water. In the example of an ammonium salt, the ammonium ion reacts with hydroxyl ions to produce ammonia and hydrogen ions, which, in turn, would lower pH. Additionally, such materials would generally tend to reduce ORP and exhibit anti-microbial properties. “Weak bases” refer to any bases where the pKb value is between 2 and 6.

In addition, strong acids (i.e., where the pKa is less than 2) may be too harmful to be used alone, but the salts thereof can be used to treat the fibers of the present disclosure. For example, salts of strong, diprotic acids, such as sodium bisulfate or sodium bisulfite, would promote low pH, since the bisulfate or bisulfite ions decompose is to form hydrogen ions, and sulfite or sulfate anions respectively.

The acids of the present disclosure may assist greatly with reducing odor in the environment in which the fibers and articles of the present disclosure are used. Many menstrual odors are alkaline in nature, regardless of the source. Use of a safe, natural acid to combat and to neutralize these agents to form less-odorous salts provides a great benefit to the user. For example, lactic acid (abbreviated as HL) could react with trimethylamine (Me₃-N) to form a protonated quaternary amine salt as follows:

H⁺L⁻+Me₃N [symbol] Me₃-NH⁺+L⁻

This reaction proceeds quickly in the aqueous environment of the vagina. Similar reactions could be written for other alkaline species causative of feminine odors. Lactic acid is particularly advantageous, as it is safe, colorless and “natural”, i.e. it is produced naturally in most healthy women. Other similar acids, such as those listed above, can also assist in this reaction. This is another advantage of the fibers of the present disclosure.

### B. Finishing (Wetting) Agent and Additional Compounds

The fibers are also treated with a second additive, namely a finishing agent, also known as a wetting agent. Most finishing agents are nonionic surfactants, but anionic surfactants can be used as well. Anionic surfactants, such as sodium lauryl sulfate, are slightly acidic and thus may act to reduce pH also. The finishing agent can be polyoxyethylene esters of fatty acids and aliphatic acids, such as Afilan™ PNS and Afilan HSG-V, ethoxylated sorbitan fatty acid esters, such as Tween 20 and Tween 80, N-cetyl-N-ethyl morpholinium ethyl sulfate; sorbitan monopalmitate; polyoxyethylene 200 castor glycerides; potassium oleate, sodium salts of tall oil fatty acids, propylene glycol, polypropylene glycol, poloxamers (such as Pluronic™ Block Copolymer surfactants), tetra-functional block copolymers based on ethylene oxide and propylene oxide (such as Tetronic™ Block Copolymer surfactants), alkylphenol ethoxylates, fatty is amine ethoxylates, phosphate esters, alcohol ethoxylates, polyalkoxylated polyethers, sodium lauryl sulfate, glycerol, glycerol monolaurate (available from Med-Chem as Lauricidin™), Sokalan™ polyacrylic dispersants, or any combinations thereof. Chemical data for these and other examples of possible fiber finishes may be found in McCutcheon's Emulsifiers & Detergents, International Edition, Glen Rock, N.J.: Manufacturing Confectioner Publication, 1981 or in the Encyclopedia of Chemical Technology, Volume 22, Surfactants and Detersive Systems, pp. 360-377. A preferred finishing agent is Tween 20, which is available commercially from Croda (UK), whose chemical name is polyoxyethylene (20) sorbitan monolaurate. Tween 20 is also known as polysorbate 20. Tween 20 is suitable for use in the present disclosure because of its high HLB value, meaning that it is hydrophilic, providing some additional absorbency benefit.

The finishing agent is present in an amount of about 0.08% to about 0.7%, or exactly 0.08% to 0.7%. The amount of finishing agent needed will depend on the particular finishing agent used, and on the nonwoven processing to be done in subsequent processing steps. In a more preferred embodiment, the finishing agent is present in an amount of about 0.25% to about 0.40%, or exactly 0.25% to exactly 0.40%. This weight range is suitable when the finishing agent is Tween 20. For a different finishing agent, such as the Afilan compounds listed above, the amount of finishing agent can be lower, such as from about 0.10% to about 0.15%, or exactly 0.10% to 0.15%. As with the acids discussed above, the weight percentages of finishing agents are based on the weight of the fiber, or in the case where there is a plurality of fibers in an absorbent article, on the weight of the total fiber content of the article

When the articles of the present disclosure are wipes, other compounds could be added. These compounds would have less of an effect on pH and ORP than those listed above, but would provide additional skin benefit, anti-bacterial benefits and/or other wellness benefits. Such compounds can be xanthan gum, PEG-40 hydrogenated castor oil, SD alcohol 40, Aloe Barbadensis leaf juice, PEG-60 lanolin, quaternium-52, PEG-8 dimethicone, sodium capryloamphoproprionate, phenoxyethanol, methylparaben, ethylparaben, propylparaben, caprylic capric triglyceride, olive tree extract, bis-PEG/PPG-16/16 PEG/PPG, rosemary oil, benzyl alcohol, propylene glycol, maltodextrin, olive leaves, chamomile extract, rosemary leaves, glycerine, fragrance formulations, or any combinations thereof.

When the articles of the present disclosure are wipes, low or very low pH fibers can often provide the added benefit of reducing the amount of preservative needed, since some of the acids and finishing agents described above can provide additional preservative benefits. The basis weight would be in the usual range for wipes, that is, about 40 to about 70 grams per square meter (gsm). The mixture of fibers used in the wipe can include polyolefins, polyester, or cellulosics, with the proviso that at least one component would be the low pH fiber of the present disclosure.

### C. Non-Acids

In addition to the acidic agents listed above, other, non-acid additives could be used to influence the pH and ORP. These include substances that produce anions and/or cations that could change valency as a result of electrochemical reactions. Examples include zinc-containing salts and compounds, e.g. zinc oxide, copper-containing salts and compounds, e.g. copper sulfate, iron-containing salts and compounds, e.g. iron sorbitex (a glucitol iron complex, compound with citric acid), potassium benzoate, sodium benzoate, trisodium citrate, ethylene diamine tetraacetic acid (and salts thereof), sodium bisulfite, sodium metabisulfite, sodium acetate, sodium propionate, potassium sorbate, sodium hypophosphite, sodium hypochlorite, potassium oxalate monohydrate, chitosan (cationic polysaccharide) salts, or any combinations thereof. These compounds can be added to the articles of the present disclosure in the same way as the acids and finishing agents discussed above. Some, for instance the is acid salts like sodium bisulfite in particular, will also lower pH. Others, like trisodium citrate, may increase pH, so other adjustments may be necessary to achieve the proper final desired pH.

Many chemicals—in particular, salts of heavy metals—might affect pH and ORP but would be excluded from consideration because of toxicity or safety. However, a wide variety of other mildly oxidizing or mildly reducing agents could be used in the body at low levels to promote wellness, often at very low levels. When the articles of the present disclosure are used intravaginally, the ORP of the vagina could be adjusted, as needed, to promote vaginal health using ingredients like those of the above. Moreover, some of these ingredients may have secondary benefits; that is, they may serve to promote other wellness functions—which may or may not be pH- or ORP-related. Secondary benefits may include skin lubrication, moisturization, sequesterization of skin irritants, odor control, heating/cooling, or aesthetics. Such ingredients would be added at levels in the 0.01-1% range.

### D. Method of Making the Fibers

The present disclosure also provides a method for making the low or very low pH fibers described above. As previously discussed, the addition of the additives listed above could be during fiber synthesis, during manufacturing of nonwoven webs comprising the fibers, during conversion processes involving those webs, or during the formation of the article in which the nonwoven webs are used.

The articles of the present disclosure comprise nonwoven webs—either rolled or folded—primarily comprising absorbent cellulosic fibers. Often, the cellulosic fiber used is rayon, whose absorbency is high and can generally be controlled well enough to meet the governmentally regulated absorbency requirements. The rayon viscose process, commonly used to make rayon, is known (see, for example, URL: http://www.mindfully.org/Plastic/Cellulose.Rayon-Fiber.htm). This reference states that, starting with wood pulp, there are 13 steps to make rayon fiber: 1) steeping, 2) pressing, 3) shredding, 4) aging, 5) xanthation, 6) dissolving, 7) ripening, 8) filtering, 9) degassing, 10) spinning, 11) drawing, 12) washing and drying, and 13) cutting, bundling and baling. To summarize, in the viscose (also known as cellulose regeneration) process used to make rayon fibers, cellulose derived from natural product sources is converted first to cellulose xanthogenate, which is dissolved in alkali to form viscose. Then, the xanthogenate is neutralized, and the viscose is coagulated, usually with acids and salts, to regenerate the insoluble cellulose. This last step is typically conducted in the presence of sulfuric acid and a zinc sulfate salt spin bath Thus, pH is ordinarily low at this stage of the process (about pH=3). The insoluble cellulose can then be extruded from the spin bath using spinnerets, to make very small fibers.

The fibers are typically stretched and cut to size and then subsequently washed. They may be bleached with agents such as sodium hypochlorite or hydrogen peroxide to adjust the fiber color and opacity. Finish agents are added and the pH is adjusted. Typically, during these steps, the pH increases to about 4. A final sour (a very dilute acid) wash is included to remove any bleaching impurities. This provides a pH of about 4 prior to drying. Usually a final wash is done to remove bleaching impurities prior to cutting. In contrast to fibers of this present invention, to make conventional rayon fibers, the pH is then usually adjusted upwards to about 6 by adding some alkaline solution at this point. The fibers are dried, bundled together, and finally packed into bales. These bales are then processed to form a nonwoven web, from which articles (e.g., the tampon or wipe) are formed.

To manufacture the fibers of the present disclosure, any of the acid additives, with or without the corresponding mineral salt, could be added during the above-described process. In one embodiment, the acid or salt would be added to adjust the pH at the end of the process, i.e. after the cellulosic fibers have been regenerated. In this embodiment, the acids, salts, or other additives could be added after step 11 and before step 13, as outlined above. In another embodiment, the acid with or without the corresponding mineral salt can be added to the fibers once they have been formed into nonwoven webs. In another embodiment, the acid with or without the corresponding mineral salt could be added during the article-forming step.

One way to add the additives would be to apply them by spraying the solution onto a thin, low basis weight nonwoven strip cut from the fiber web during the tampon forming process. This additional nonwoven strip (which could also be a fibrous felt or foam) could then be combined with the rest of the cellulosic-based web piece(s). Then, the webs could be rolled or folded up and finally compressed make the actual tampon pledget. In another embodiment, the acid with or without the corresponding mineral salt is added at some point during the viscose process, for example while the viscose is being coagulated.

There is a limit on how low pH can go in cellulose fiber manufacturing. In the rayon viscose process described above, the pH increases during the bleaching and washing steps, so pH is typically lower prior to these steps. If the cellulose fiber remains at a pH of 2 or less for too long, however, the cellulose can begin to decompose, causing fiber quality problems (e.g. low wet strength and/or disintegration of the fiber into powder). Thus, pH after the coagulation step is in the 2.5 to 3 range, from which point it increases to around 4 during bleaching and washing. When adding the acid or salts thereof during the viscose process, it is important to meet these guidelines.

Because of this, it was previously not thought possible or advisable to add an acid to the manufacturing process, such as that described in the present disclosure. It was previously thought that the acid would lower the pH of the cellulose processing mixture, and adversely affect the final product. According to the present disclosure, however, a rayon fiber pH target of 4 can be produced at high quality, and this is close to the optimal pH required to avoid problems of pathogenic bacteria in the vagina. Clinical studies suggest that a pH in the vaginal area significantly below 3 may actually cause wellness problems; moreover, very low pH values can cause the cellulosic fiber to disintegrate into powder and thus be ineffective in end-use applications such as absorbent tampons.

Besides the viscose process, there are other methods used to make cellulosic fibers, such as the N-methyl N-morpholine slurry process used to make Tencel fibers (as sold by Lenzing, in Austria). As with the viscose process, the pH of such fibers could be adjusted to be lower and to provide a greater health benefit.

During web processing, a variety of agents could be added to lower pH and/or influence ORP. These would include latex (chemical) bonding agents. Often, to bind/entangle webs together for subsequent tampon forming, latex binders are used. These are typically polymeric binders made of acrylic polymers, vinyl acetate polymers, olefinic polymers or styrene-butadiene polymers. An example of a suitable binder is Rhoplex™ NW1402, available from Dow Chemicals' Rohm and Haas Division (Midland, Mich.). Generally, these are aqueous based, synthetic systems, so the pH could be adjusted at any point by addition of acids and/or electrolytes during their manufacturing processes. The latex binders adhere to fibers, acting as adhesive promoters to ensure that the fibers remain tightly bonded to one another. They would be added during the nonwoven processing step described above.

In some cases, during web processing, web conversion steps are taken. Web conversions may include, for example, printing, decorating, embossing, and the like. During these processes—which may involve other fibers, inks, or mechanical or chemical treatments—the additives discussed above could be added to affect the final article pH and ORP.

### E. Fiber Characteristics

Rayon having a multilobal (i.e. a “Y” shape cross-section) morphology provides superior absorbency when this rayon is used in a menstrual tampon. (See, e.g., U.S. Pat. Nos. 5,634,914, and 6,333,108, both to Wilkes et al.) This fiber is available commercially as Galaxy™ from Kelheim (Kelheim, Germany). The present disclosure has discovered that the Galaxy fiber, when treated with lactic acid, can be formed into an article that not only provides superior absorbency in a tampon, but also promotes wellness by providing a low pH environment during women's menstrual periods. This characteristic Y shape is obtained by extruding the fibers through the spinneret that has Y-shaped dies. The multilobal morphology provides an advantage over other shapes, such as normal viscose, whose cross-sectional shape is roughly cylindrical (as opposed to Y shaped).

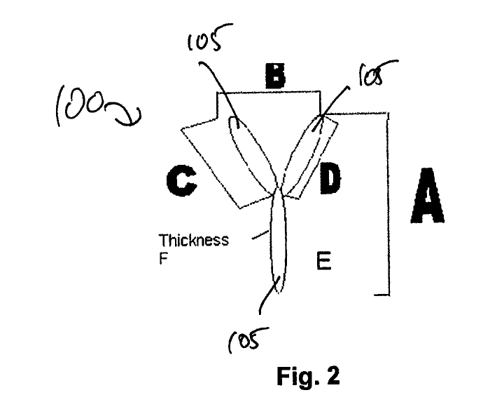

FIG. 2 shows a schematic cross-sectional drawing of a multilobal fiber 100. FIG. 2 provides the preferred geometry of the fiber of this invention obtained from precision optical microscopy and is similar to that revealed in the patents referenced above. Fiber 100 has three branches 105, having lengths C, D, and E, and an effective diameter A. In one embodiment, the ratio of A:C:D:E can be about 1.0:0.7:0.7:0.5, where A is between about 20 to about 50 microns. The thickness F of each of branches 105 is very hard to measure accurately from micrographs, but its ratio relative to the distance A can be about 0.185:1.

In one embodiment, the fiber used to make the articles of the present disclosure comprises a cellulosic blend of cotton and rayon, which comprises at least 92% cellulose by weight. In one embodiment, the fiber used to make the articles of the present disclosure comprises 98% to 99.5% of a cellulosic fiber, such as multilobal rayon.

### F. Encapsulating Agents

The present disclosure further contemplates that various agents could be used together with the acids and/or finishing agents listed above to deliver the acids and finishing agents to either the vaginal area or the skin (where the article of the present disclosure is a tampon or a wipe, respectively) in a more effective, time-release manner. One class of agents that could achieve this function is encapsulating agents. Examples of encapsulating agents include cyclodextrins, which are large, “caged” compounds is often used to bind to and/or encapsulate smaller molecules. Cavitron™ cyclodextrins (American Maize-Products Company, IN) are one example. Zeolites are another “caged” compound that releases ingredients via a controlled-release, ion exchange type method. Tiny microcapsules, either made from gelatin or derived from plant sources, can also be used to encapsulate the acids and finishing agents of the present disclosure. Theis Technology produces a variety of coated capsules for this purpose. Methocel™ can be used to deliver the acids and finishing agents as well as to bind/adhere them to fibers or webs used in tampons. VegiCaps Soft capsules from Cardinal Health or EcoCaps from Banner Pharmacaps (NC), which are based on a seaweed extract, are also suitable encapsulating agents. Encapsulence® advanced microencapsulation technology (Ciba) is another approach that could be used to encapsulate these ingredients used in pH and/or ORP control.

Nanotechnology advances could also be leveraged in binding and encapsulation. One such nanotechnology advance was recently described by Dr. Joseph M. DeSimone and coworkers at the University of North Carolina at Chapel Hill (JACS, Jul. 20, 2005) and is known as “liquid Teflon”. This material is used to make molds to craft particles that in turn carry active ingredients (i.e. pH and/or ORP controlling ingredients) as “cargo” to a specific source. Another advance is known as MicroPlant (Q-Chip, UK). This is a fully-functioning microcapsule development platform that uses microfluidic technology to control chemical reactions and to make capsules of various sizes for controlled release purposes.

### G. Experimental Data

Table 1 below shows the calculated pH for aqueous extractions from a fiber of the present disclosure. In the shown examples, the acid is lactic acid, and the mineral salt is sodium lactate. The data shows that adding the lactic acid with or without the sodium lactate, in the amounts recited above in Section A, provide an aqueous extraction with a pH that is in the desired range for vaginal wellness.

| | % grams | Weight % | Weight % of both | | |
|---|---|---|---|---|---|
| | of lactic | sodium | sodium lactate and | | |
| | acid on | lactate on | lactic acid | Computed | |
| Calculation | fiber | fiber | on fiber | pH | Comment |
| 1 | 0.040% | 0.040% | 0.080% | 4.531 | added acid and |
| 2 | 0.040% | 0.000% | 0.040% | 4.459 | mineral salt in same |
| 3 | 0.040% | 0.050% | 0.090% | 4.548 | weight proportions |
| 4 | 0.040% | 0.020% | 0.060% | 4.496 | no added lactate |
| | | | | | buffer |
| | | | | | added acid and |
| | | | | | mineral salt in same |
| | | | | | molar proportions |
| | | | | | added sodium lactate |
| | | | | | one-half of that of |
| | | | | | lactic acid |
| 5 | 0.200% | 0.200% | 0.400% | 4.124 | added acid and |
| 6 | 0.200% | 0.000% | 0.200% | 3.934 | mineral salt in same |
| 7 | 0.200% | 0.249% | 0.449% | 4.163 | weight proportions |
| 8 | 0.200% | 0.100% | 0.300% | 4.034 | no added lactate |
| | | | | | buffer |
| | | | | | added acid and |
| | | | | | mineral salt in same |
| | | | | | molar proportions |
| | | | | | added sodium lactate |
| | | | | | one-half of that of |
| | | | | | lactic acid |
| 9 | 0.400% | 0.400% | 0.800% | 4.005 | added acid and |
| 10 | 0.400% | 0.000% | 0.400% | 3.737 | mineral salt in same |
| 11 | 0.400% | 0.498% | 0.898% | 4.058 | weight proportions |
| 12 | 0.400% | 0.200% | 0.600% | 3.882 | no added lactate |
| | | | | | buffer |
| | | | | | added acid and |
| | | | | | mineral salt in same |
| | | | | | molar proportions |
| | | | | | added sodium lactate |
| | | | | | one-half of that of |
| | | | | | lactic acid |

Table 2 shows the amounts of acid and finishing agent present in several different fibers and articles of the present disclosure. Table 2 also compares these amounts to a tampon having fibers with a higher pH. In Table 2, the acid is lactic acid, and the finishing agent is Tween 20. The lactic acid levels provided in Table 2 include both the fully protonated lactic acid, as well as any lactate ions that may have been added together with the lactic acid as a buffering agent, in the form of the mineral salt.

TABLE 2

| | Average Tween | Average Lactic |
|---|---|---|
| | 20 (% based | acid (% based |
| | on total | on total |
| Sample Description | fiber) | fiber) |
| Very Low pH (Target: 3.8) Fiber | 0.228% | 0.670% |
| Low pH (Target: 4.2) Fiber | 0.199% | 0.120% |
| Web Made from Low pH (Target: 4.2) | 0.315% | 0.070% |
| Fiber | | |
| Tampons Made from Low pH (Target: | 0.100% | 0.150% |
| 4.2) Fiber/Web | | |
| Control Tampons Made Using Higher | 0.311% | N/A (0) |
| pH (Target: about 6) Fiber | | |
| Pooled Standard Deviation Estimate | 0.07% | 0.02% |

The Tween 20 nonionic surfactant levels range widely, from a low of 0.10% to a high of 0.43%. This table only shows averages of duplicate determinations, but the standard deviations are high, particularly for Tween 20, likely due to both a combination of errors due to Tween 20 distribution on fiber and analytical error.

Lactic acid is much higher for the very low pH (target: 3.8) fiber than for the low pH (4.2) fiber, webs and tampons. Also, presumably because of some washing treatments performed in webbing and (subsequently) forming the tampons, some of the lactic acid is diluted to even lower levels for the 4.2 pH webs and tampons. This is partly because of the nonlinearity associated with pH and concentration of the lactic acid, as illustrated in FIG. 1. FIG. 1, which provides calculated values of pH based upon treatment of fibers with lactic acid, shows why the preferred level of lactic acid is about 0.30-0.65%, which is high enough to maintain a low, stable pH, but sufficiently low so as to not interfere with other properties. (See Syngyna absorbency results, as discussed below.)

Table 3 below shows the results of an absorbency test conducted on two sets of bagged tampons made in the laboratory. One set was made from the low pH fibers of the present disclosure, the other from a more standard, higher pH fiber. Both sets of bagged tampons were made according to the Instructions outlined in Test Method I below. They were evaluated for Syngyna absorbency, Test Method V below, and the pH was measured in accordance with Test Method IV below. 20 tampons were made and evaluated for Syngyna absorbency for each of the 4 cells outlined below. 2 tampons for each of the 4 cells were evaluated for pH. The effect of combing and carding the fiber was not significant.

TABLE 3

| | Absolute | Gram per | |
|---|---|---|---|
| | Syngyna | gram Syngyna | |
| Type of Fiber Evaluated | Absorbency | Absorbency | pH |
| Low pH fiber (variant) | 9.144 | 2.881 | 4.103 |
| High pH fiber (control) | 9.440 | 2.930 | 6.025 |
| Standard deviation of estimate | 0.253 | 0.081 | 0.016 |

As is shown, the two samples (the controls and variants, i.e. the fibers of the present disclosure) exhibited nearly equivalent Syngyna absorbency test results, is whether absolute or gram per gram. With bagged tampons, results are more variable than with standard tampons made using the more conventional webbing and forming procedures described below, but there is a very clear difference in pH for the fiber of the present disclosure as compared to the control.

For the data shown in Tables 4 and 5 below, a larger batch of low pH fiber was tested, so that the samples could be prepared according to a method that more closely approximates large-scale commercial production, according to Method III below. For these tests, the appropriate control was commercially available Sport Super® unscented tampons.

In all, there were five “cells” for comparisons. Table 4 provides the identification of these five cells. Cells 1 and 4 are considered controls, since standard fiber was used, whereas cells 2, 3 and 5 are to be considered as low pH fiber variants. Some 40,000 tampons were produced at commercial scale based on these low pH fibers. There were no problems in either making the webs or in forming these tampons.

TABLE 4
Cell Identifications

Cell 1 = Lab-made, Sport Super, made from control, standard
fiber made into webs
Cell 2 = Lab formed, Sport super, low pH fiber bale, web not
pre-conditioned in the humidity chamber
Cell 3 = lab formed, Sport, low pH fiber bale, web pieced
pre-conditioned in the humidity chamber
Cell 4 = Sport controls, i.e. commercial tampons
Cell 5 = Sport super tampons, commercial scale, low pH fiber
bale

Table 5 lists the key results from the evaluation of tampons from these cells, using the test methods outlined in Sections V and VI below.

TABLE 5

| | | gram per gram Syngyna | Ejection |
|---|---|---|---|
| Cell | Low or High pH | Absorbency | Force, oz. |
| 1 | high pH control | 4.040 | 10.00 |
| 2 | low pH | 3.990 | 10.29 |
| 3 | low pH | 4.047 | 10.00 |
| 4 | high pH control | 3.872 | 11.32 |
| 5 | low pH | 4.045 | 13.17 |

| | | |
|---|---|---|
| Standard Error of Estimate | 0.090 | 0.332 |

As is shown in Table 5, there is some variation in gram per gram absorbency results and in ejection forces, but the results, for the most part, suggest equivalent performance in these key tampon performance characteristics.

Absorbency testing for cell 5 has been repeated several times since the initial results were obtained. The result for an average of 25 Syngyna results was 3.94 grams per gram, consistent with the results provided in Table 4. In general, tampons made from low pH fiber are just as stable, just as absorbent and perform just as well as tampons made from standard pH fiber. The average pH for the tampons from cell 5 was 4.2, with a range observed from 4.0 to 4.4. The average observed for control tampons in cell 4 was 5.8. Measurements were done according to test methods outlined in Section IV below.

A zone of inhibition test was also conducted to determine whether extracts from the low pH fiber would affect vaginal flora. Section VII below details how this test is performed. It was conducted on tampon samples made from both standard and low pH fibers. Results were negative in both series of tests, indicating that there is no adverse influence on vaginal flora.

An additional set of data is shown in Table 6-8. Tampons were made from a batch of very low pH (target 3.8) fiber and compared with suitable controls. In these examples, tampons bagged directly from fiber, tampons in production equipment, and tampons made in the laboratory from webs made in production, were all prepared according to previously discussed methods.

TABLE 6

| Example | Fiber/Tampon Used | No. of Tampons Made and Process Used |
|---|---|---|
| C-A | Standard Sport Super | None made in lab. 30 commercial tampons |
| | Unscented, commercial | collected for testing and comparison purposes. |
| | tampons. Standard Galaxy | |
| | Fiber. | |
| E-A | Tampons. Made in a special | None made in lab. 40,000 made in plant trial. 30 o [symbol] |
| | trial with Low pH (3.8) | these tampons were collected for testing and |
| | multilobal fiber. | comparison purposes. |
| E-B | Tampons. Made in a special | 20,000 made in plant trial. 30 of these “digital” |
| | trial with Low pH (3.8) | tampons were collected, strung in the lab, and the [symbol] |
| | multilobal fiber. No | used for testing and comparison |
| | applicator: digital. | |
| C-B | Standard Multilobal Galaxy | 35+ formed in lab, according to previously |
| | Fiber Webs. Tampons Made | described procedure. |
| | in Lab. | |
| E-C | Low pH (3.8) Multilobal | 35+ formed in lab, according to previously |
| | Galaxy Fiber, formed into | described procedure. |
| | Webs. Tampons Made in | |
| | Lab. | |
| C-C | Standard Multilobal Galaxy | 25 formed in lab, using bag pledget process |
| | Fiber. Bag pledgets. Lab | previously described. |
| | prepared. | |
| E-D | Low pH (3.8), multilobal | 25 formed in lab, using bag pledget process |
| | Fiber. Bag pledgets. Lab | previously described. |
| | prepared. | |

[symbol]

indicates data missing or illegible when filed

TABLE 7

| | | | | | | | Ejection Force |
|---|---|---|---|---|---|---|---|
| | | | | | Gram per | | (1 week, in an |
| | | | | | gram | Ejection | environmental |
| | | Dry Weight, | Absolute | | absorbency, | Force | Chamber set at 78 |
| | Brief | Avg. of 10 | Absorbency, | Moisture, | computed, | (initial), | deg F., 75% R.H.), |
| Example | Description | samples | Avg. of 10 | avg. of 4 | avg. of 10 | avg of 10 | avg. of 10 |
| C-A | Std. Sport Super | 2.73 | 10.83 | 11.33 | 3.85 | 10.14 | 12.38 |
| E-A | 3.8 pH, | 2.75 | 10.15 | 11.93 | 3.60 | 12.89 | 13.42 |
| | Production * | | | | | | |
| E-B | 3.8 pH, | | | | | | |
| | Production. | 2.76 | 10.10 | 12.38 | 3.59 | N/A | N/A |
| | Digital | | | | | | |
| C-B | std web, lab prep | 2.81 | 11.00 | 14.00 | 3.91 | 10.21 | 11.85 |
| E-C | low pH (3.8) | 2.79 | 10.52 | 13.43 | 3.74 | 10.32 | 11.55 |
| | web, lab prep | | | | | | |
| C-C | Std. Fiber, | 2.93 | 7.52 | 10.67 | 2.47 | N/A | N/A |
| | bagged | | | | | | |
| E-D | 3.8 pH fiber, | 2.93 | 7.25 | 10.69 | 2.38 | N/A | N/A |
| | bagged | | | | | | |

| | | | | | | |
|---|---|---|---|---|---|---|
| Pooled Std. Error of Estimates | 0.010 | 0.111 | 0.082 | 0.037 | 0.346 | 0.781 |

Table 6 provides a summary of the comparative examples. Table 7 provides a summary of the Syngyna absorbencies, moistures, ejection forces, and ejection forces after subjection to an environmental chamber. As Table 7 shows, for tampons made using the very low pH fiber (samples E-A, E-B, E-C, and E-D) there is a slight lowering in Syngyna absorbency, when compared to the control samples (C-A, C-B, and C-C). As shown in Table 2 above, the samples using the very low pH fiber have a significant amount of lactic acid present, approximately 0.67% based on the total fiber weight, which likely affects the much more absorbent cellulosic portion of the fiber. This lower is pH had not been observed for the low pH (target: 4.2) tampons, discussed in Tables 3-5, for which the lactic acid level was much lower (0.15%, based on the total weight of the fiber). Thus, to strike a balance between the two, sometimes competing interests, namely low pH and absorbency, it is desirable to have loading of the acid somewhere between the points of 0.15% and 0.67%, for example at about 0.60%. (See FIG. 1.)

For sample E-A, 33 additional samples were tested. These results are as follows: absolute absorbency 10.08 g, moisture 10.7%, initial ejection force 12.6 oz. This confirms that there is only a slight drop off in absorbency when the very low pH fibers of the present disclosure are used to make tampons.

TABLE 8

| | | | ORP, mv., |
|---|---|---|---|
| | | pH, Avg. of 4 | Average of 2 |
| | | measurements | measurements |
| | Brief | for fiber from | for fiber from |
| Example | Description | tampons | tampons |
| C-A | Std. Sport Super | 6.48 | 325.25 |
| E-A | 3.8 pH, | 3.68 | 492.55 |
| | Production * | | |
| E-B | 3.8 pH, | 3.72 | 546.05 |
| | Production. | | |
| | Digital | | |
| C-B | std web, lab prep | 6.25 | 367.90 |
| E-C | low pH web, lab | 3.76 | 421.10 |
| | prep | | |
| C-C | Std. Fiber, | 6.56 | 447.45 |
| | bagged | | |
| E-D | 3.8 pH fiber, | 3.75 | 464.65 |
| | bagged | | |

| | | |
|---|---|---|
| Pooled Std. Error of Estimates | 0.081 | 21.38 |

Table 8 provides pH and ORP results for the samples discussed in Tables 6 and 7. Test methods for these measurements appear below. This consistency is to be expected, given the plateau observed in pH at high lactic acid concentrations, as shown in FIG. 1. Although the ORP value is raised slightly with samples E-A and E-B., which are made with the low pH fibers of the present disclosure, the ORP values for these samples are still well within what would be considered acceptable ranges for vaginal wellness. The fibers of the present disclosure therefore provide both low pH and satisfactory ORP readings.

### H. Testing Methods

The following testing methods were used on the samples discussed above.

### I. Test Methods for New Fiber Evaluation by the Bagged Pledget Method

Several groups of tampons comprising bagged fiber pledgets were made for testing the fibers of the present disclosure. A “pledget” is the compressed and heated fiber bundle that is commonly known as a tampon. Pledgets comprising four different fiber samples were made, namely (1) uncombed, standard pH fiber, (2) uncombed, low pH fiber of the present disclosure, (3) combed and carded standard pH fiber, and (4) combed and carded low pH fiber of the present disclosure. Pledgets of each of these fibers were placed in bags according to Method II described below, and tested, as described earlier.

### II. Methods for Making Coverstock Bags Needed for New Fiber Evaluations

The coverstock used for the bags described above in Method I can be, for example, a spunbond polyethylene/polyester heat-sealable nonwoven blend, 16 gsm, available from HDK Industries (SC), cut using the automated cutter (Sur-Size™, Model # SS-6/JS/SP, available from Azco Corp., NJ). The coverstock should be cut into appropriately sized pieces, which in the present disclosure was 4.5″×3.75″. These pieces can be formed into bags by an ultrasonic device that heats the stock and seals it to itself.

### III. Standard Procedure for Making Tampons Using the HP Simulator

First, nonwoven webs are made by using a Rando webber (Rando Machines, NY). A needle punching machine is used to form and bind the appropriate nonwoven webs together. The webs are cut into strips, placed in a cross-pad configuration, compressed, and heated, to form the pledget. The pledget is threaded with a string, and placed in an applicator. This process more closely approximates large-scale production, as compared to Method I described above.

### IV. Method for Potentiometric Determination of pH and ORP in Aqueous Extract of Tampon Fibers

A good quality pH Meter (e.g. Orion Model 701A or equivalent) is used for these measurements. The combination pH electrode is Orion “Ionanlyzer” # 91-04-00 (or an equivalent electrode). A 1% saline solution is prepared, and adjusted to 7.0 pH. 1.00 g of fiber is weighed into a 250 ml beaker. 100 ml of the 1% saline solution is added. The beaker is covered, and stirred with a magnetic stirring system, for 5 minutes. The temperature is adjusted to exactly 25 deg C. The mass of fiber is removed from the beaker, and the pH of the remaining solution is measured.

ORP measurements reported above were determined using a similar method with the same pH Meter, but with a special Fisher ORP electrode to measure the oxidation-reduction potential in millivolts. The electrode used was Model # 13-620-81, a Pt/Ag/AgCl combination electrode.

### V. Syngyna Test Method (Absorbent Capacity)

Testing is done, in accordance with Standard FDA Syngyna capacity as outlined in the Federal Register Part 801, 801.43.

### VI. Ejection Forces

Tampon ejection force is measured in the laboratory by a special test. The assembled tampon is gripped using two fingers on either side of the fingergrip on the barrel of the applicator. The force in ounces exerted on a high precision weighing scale (a Weightronix WI-130 load cell) to eject the pledget is then measured.

### VII. Agar Diffusion Zone of Inhibition: Vaginal Flora

This microbiological test method tests whether or not materials would affect vaginal flora adversely or not. The test was conducted according to what is disclosed in US Pharmacopeia, “Biological Tests and Assays <81>, USP 26, NF 21. Paper discs are placed onto glass microscope slides. Aqueous extract solutions from the fibers to be tested are prepared, and applied to the discs. Mixtures of lactobacillus cultures that are typically found in human vaginas are prepared, and swabbed onto agar plates, to which the paper discs are then added.

The presence of a clear zone around the discs indicates a positive zone of inhibition, demonstrating the sample material has the ability to alter the growth of the microorganisms. Absence of a clear zone around the discs indicates a negative zone of is inhibition, demonstrating the sample material does not have the ability to alter the growth of the microorganisms.

### G. Additional Optional Embodiments and Components

Other absorbent fibers such as cotton, also used in tampons, would involve similar but simpler processes for pH adjustment. Cotton is usually preprocessed to remove non-cellulosic impurities and then bleached. During or after bleaching, its pH could also be adjusted during washing to increase it to the 3 to 4 range before drying. Similarly, Lyocell™ and Tencel™—two rayon grades made using a solvent/slurry process with N-Methylmorpholine N-oxide (NMNO)—could also be post-treated with pH-reducing agents. For reasons similar to those outlined above for the viscose products, it is possible to achieve a minimum pH of about 4, but difficult to go below this, due to loss of fiber strength and integrity.

Besides cellulosic fibers, acid-containing fibers could be used to affect pH and/or ORP. For example, superabsorbent fibers could be used in a partially neutralized state to lower pH. Oasis™ fibers (Technical Absorbent Products, UK) or Camelot™ fibers (CA) are comprised of polyacrylic acid/sodium polyacrylate. By adjusting the level of fiber in the tampons and/or by adjusting the acid/salt balance by only partially neutralizing the polyacrylic acid in these fibers, one could influence both pH and ORP.

Another approach would be to use antibacterial fibers. For example, Healthgard Rayon, Zincfresh Viscose (Lenzing, AU) or Tencel Silver (Lenzing, AU) are all approaches which use electrolytes incorporated into rayon-based fibers, thus influencing ORP. These have been demonstrated to be effective against pathogenic bacteria and could be used in tampons.

Tampons are usually covered with a thin strip of coverstock material. ORP and/or pH controlling agents could be added together (with or without finish) to coverstock in a manner similar to that described above.

Another way might be to spray or dip a solution containing finish, pH and/or ORP controlling agents directly onto a plastic or cardboard applicator. Once the tampon has been formed, some extraction of the more hydrophilic components would take place onto the pledget (from the inside of the applicator) or into the vagina (from the outside of the applicator).

There are also a variety of different agents that one could use to deliver pH and/or ORP controlling agents to tampons. One such class includes functionalized particles. Different sized particles (from submicron to millimeter sized) can be made using (micro) suspension polymerization techniques and functionalized as required by an application. One class of particles would include superabsorbent particles. Like the superabsorbent fibers mentioned earlier, these particles could be only partially neutralized, to provide a lower pH in the tampons. These particles could be mechanically or chemically bound to the cellulosic fibers and/or added using bags made of other fibers.

Acids and other additives could be incorporated into polyacrylate spheres, making use of a micro sponge technology, for time-release benefit. Another type of particle used to deliver pH and/or ORP controlling ingredients would be DispersEZ fluoro particles. Usually these particles are supplied as a suspension in water. Acid or electrolyte moieties could be added to the surface of these particles and released as needed.

Still another approach would be to use Cavilink™ polymers (Sunstorm Technologies, Calif.). These are porous polymeric spheres that would allow a variety of different agents to be added to tampons. These act as sort of “microsponges”. Ingredients could be added either through the pores and/or by means of functionalizing the surfaces of these particles.

Liquid encapsulants could also be used to deliver pH and/or ORP controlling ingredients. Examples of these might include LoSTRESS™ liquid encapsulants (Polysciences, PA).

Still another approach would be to use biodegradable implants that degrade slowly to release pH and/or ORP ingredients slowly to the body. One way to do this would be to use Durin™ implants (Durect) designed to degrade over many weeks. The biodegradable polymer used is poly(DL-lactide-go-glycolide) which degrades in body to lactic acid and glycolic acid. This could be added to the tampon in fiber, web or particulate form. Chemically polylactide polymers (available from EarthWorks LLC, a division of Dow Cargill, NE) are quite similar, biodegradable materials, which have been fashioned into Ingeo™ fibers (Ingeo, Minn.).

Finally, to measure pH and ORP, a combination pH-ORP electrode could likely be fashioned to resemble a tampon, in order to carry out measurements of both of these key quantities. This would allow direct, in-body, real-time control of these two quantities. The amount applied to the tampon could then be manipulated/adjusted to promote wellness most effectively.

The present disclosure has been described with particular reference to several embodiments. It should be understood that the foregoing descriptions and examples are only illustrative of the invention. Various alternatives and modifications thereof can be devised by those skilled in the art without departing from the spirit and scope of the present disclosure. Accordingly, the present disclosure is intended to embrace all such alternatives, modifications, and variations that fall within the scope of the appended claims.

What is claimed is:

1. A low pH fiber, comprising:
about 0.05% to about 0.8%, based on the weight of the fiber, of a first additive selected from the group consisting of citric acid, lactic acid, isoascorbic acid, glycolic acid, malic acid, tartaric acid, glycolide, acetic acid, dehydroacetic acid, boric acid, oleic acid, palmitic acid, stearic acid, behenic acid, palm kernal acid, tallow acid, salicylic acid, ascorbic acid, sorbic acid, benzoic acid, succinic acid, acrylic acid, and any combinations thereof; and
about 0.08% to about 0.7%, based on the weight of the fiber, of a second additive selected from the group consisting of polyoxyethylene esters of fatty acids and aliphatic acids, ethoxylated sorbitan fatty acid esters, N-cetyl-N-ethyl morpholinium ethyl sulfate, sorbitan monopalmitate, polyoxyethylene 200 castor glycerides, potassium oleate, sodium salts of tall oil fatty acids, propylene glycol, polypropylene glycol, is poloxamers, tetra-functional block copolymers based on ethylene oxide and propylene oxide, alkylphenol ethoxylates, fatty amine ethoxylates, phosphate esters, alcohol ethoxylates, polyalkoxylated polyethers, sodium lauryl sulfate, glycerol, polyacrylic dispersants, and any combinations thereof.

2. The low pH fiber of claim 1, wherein said first additive is an acid selected from the group consisting of citric acid, lactic acid, isoascorbic acid, and any combinations thereof.

3. The low pH fiber of claim 2, further comprising a mineral salt of said acid.

4. The low pH fiber of claim 1, wherein said second additive is polysorbate 20.

5. The low pH fiber of claim 1, wherein said first additive is present in an amount of about 0.30% to about 0.65%, based on the weight of the fiber.

6. The low pH fiber of claim 1, wherein said second additive is polysorbate 20 and is present in an amount of about 0.25% to about 0.40%, based on the weight of the fiber.

7. The low pH fiber of claim 1, wherein an aqueous extract of the low pH fiber has a pH between about 3.5 and about 4.7.

8. The low pH fiber of claim 1, wherein an aqueous extract of the low pH fiber has a pH between about 3.7 and about 3.9.

9. A fibrous article, comprising:
a plurality of low pH fibers,
about 0.30% to about 0.65%, based on the weight of said plurality of low pH fibers, of a first additive selected from the group consisting of citric acid, lactic acid, isoascorbic acid, glycolic acid, malic acid, tartaric acid, glycolide, acetic acid, dehydroacetic acid, boric acid, oleic acid, palmitic acid, stearic acid, behenic acid, palm kernal acid, tallow acid, salicylic acid, ascorbic acid, sorbic acid, benzoic acid, succinic acid, acrylic acid, any salts thereof, and any combinations thereof;
about 0.08% to about 0.7%, based on the weight of said plurality of low pH fibers, of a second additive selected from the group consisting of polyoxyethylene esters of fatty acids and aliphatic acids, ethoxylated sorbitan fatty acid esters, N-cetyl-N-ethyl morpholinium ethyl sulfate, sorbitan monopalmitate, polyoxyethylene 200 castor glycerides, potassium oleate, sodium salts of tall oil fatty acids, propylene glycol, polypropylene glycol, poloxamers, tetra-functional block copolymers based on ethylene oxide and propylene oxide, alkylphenol ethoxylates, fatty amine ethoxylates, phosphate esters, alcohol ethoxylates, polyalkoxylated polyethers, sodium lauryl sulfate, glycerol, polyacrylic dispersants, and any combinations thereof.

10. The fibrous article of claim 9, wherein the article is a tampon.

11. The fibrous article of claim 9, wherein the article is a wipe.

12. The fibrous article of claim 9, wherein said first additive is lactic acid.

13. The fibrous article of claim 12, further comprising a mineral salt of lactic acid, so that said lactic acid and said lactic acid are present in an amount of about 0.40% to about 0.60%, based on the weight of said plurality of low pH fibers.

14. The fibrous article of claim 13, wherein said mineral salt of lactic acid is selected from the group consisting of sodium lactate, potassium lactate, and a combination thereof.

15. The fibrous article of claim 13, wherein said second additive is polysorbate 20, and is present in an amount of about 0.25% to about 0.40%, based on the weight of said plurality of fibers.

16. The fibrous article of claim 9, wherein said plurality of low pH fibers have an aqueous extract that has a pH between about 3.5 and about 4.7

17. The fibrous article of claim 9, wherein said plurality of low pH fibers have an aqueous extract that has a pH between about 3.7 and about 3.9.

18. A process for making a low pH fiber, comprising the steps of:
converting natural cellulose to cellulose xanthogenate;
dissolving said cellulose xanthogenate in alkali, to form a colloidal viscose;
coagulating said colloidal viscose;
drying said colloidal viscose;
drawing a fiber from said dried colloidal viscose; and
adding an acid selected from the group consisting of citric acid, lactic acid, isoascorbic acid, and any combinations thereof, to either said colloidal viscose, or said fiber, to form said low pH fiber.

19. The process of claim 18, wherein said acid is added during said coagulation step.

20. The process of claim 18, further comprising the step of forming a nonwoven web from said plurality of said moderately low pH fibers, wherein said acid is added to said web, to form a wed of the low pH fiber.
